(19) United States
(12) Patent Application Publication
(10) Pub. No.: US 2019/0127692 A1
(43) Pub. Date: May 2, 2019

(54) Production Method For Hepatocyte Lineage Cells, Hepatocyte Lineage Cell Or Culture Product Obtained By The Production Method, And Hepatocyte Differentiation-Inducing Medium

(71) Applicant: National University Corporation Chiba University, Chiba (JP)

(72) Inventor: Minoru Tomizawa, Chiba (JP)

(21) Appl. No.: 16/172,936

(22) Filed: Oct. 29, 2018

(30) Foreign Application Priority Data
Oct. 31, 2017 (JP) 2017-211334

(51) Int. Cl. C12N 5/071; C12N 5/074; C12N 5/00

(52) U.S. Cl. CPC C12N 5/067; C12N 5/0696; C12N 5/0607; C12N 5/0018; C12N 2506/03; C12N 2500/60; C12N 2500/34; C12N 2501/999; C12N 2500/14; C12N 2500/32; C12N 2506/45

(57) ABSTRACT

Provided are a production method for hepatocyte lineage cells, a hepatocyte lineage cell or culture product obtained by the production method, and a hepatocyte differentiation-inducing medium. It has been found that the differentiation of iPS cells to hepatocyte lineage cells is efficiently induced by adding lactic acid or a salt thereof to a related-art hepatocyte differentiation-inducing medium.

### BACKGROUND OF THE INVENTION

### 1. Field of the Invention

The present invention relates to a production method for hepatocyte lineage cells, a hepatocyte lineage cell or culture product obtained by the production method, and a hepatocyte differentiation-inducing medium.

The present application claims priority from Japanese Patent Application No. 2017-211334, which is incorporated herein by reference.

### 2. Description of the Related Art

There is a report on a method of obtaining liver from human iPS cells (Takebe, T. et al., “Vascularized and functional human liver from a iPSC-derived organ bud transplantation,” Nature, 2013, Vol. 499, p. 481-489). This method is a method of forming liver, involving promoting differentiation to hepatocytes in vitro and transplanting a mixture of the resultant with vascular endothelial cells and mesenchymal stem cells to a mouse. In Takebe, T. et al. , “Vascularized and functional human liver from a iPSC-derived organ bud transplantation,” Nature, 2013, Vol. 499, p. 481-489, the differentiation to hepatocytes is induced in accordance with a method described in Si-Tayeb, K. et al., “Highly efficient generation of human hepatocyte-like cells from induced pluripotent stem cells,” Hepatology, 2010, Vol. 51, p. 297-305. The method of Si-Tayeb, K. et al., “Highly efficient generation of human hepatocyte-like cells from induced pluripotent stem cells,” Hepatology, 2010, Vol. 51, p. 297-305 takes several weeks, and cannot provide mature hepatocytes (Si-Tayeb, K. et al., “Highly efficient generation of human hepatocyte-like cells from induced pluripotent stem cells,” Hepatology, 2010, Vol. 51, p. 297-305).

The inventors of the present invention developed a method of collecting only human primary hepatocytes by co-culturing human iPS cells and human primary hepatocytes to kill the human iPS cells (Tomizawa, M. et al., “Survival of primary human hepatocytes and death of induced pluripotent stem cells in media lacking glucose and arginine,” PLoS One, 2013, Vol. 8, e71897).

As a result of an investigation on conditions under which iPS cells survive for a long period of time, it has been revealed that addition of low-molecular-weight compounds, including an apoptosis inhibitor, and oncostatin M allows the cells to survive for up to 7 days, though in small number (Tomizawa, M. et al., “An optimal medium supplementation regimen for initiation of hepatocyte differentiation in human induced pluripotent stem cells,” Journal of Cellular Biochemistry), 2015, Vol. 116, No. 8, p. 1479-1489).

### SUMMARY OF THE INVENTION

It is an object of the present invention to provide a production method for hepatocyte lineage cells, a hepatocyte lineage cell or culture product obtained by the production method, and a hepatocyte differentiation-inducing medium.

In order to achieve the above-mentioned object, the inventors of the present invention have made extensive investigations on factors (in particular, deficient nutrient sources) for initiating the differentiation of iPS cells serving as pluripotent stem cells to hepatocytes. As a result, the inventors have found that the differentiation of iPS cells to hepatocyte lineage cells is efficiently induced by adding lactic acid or a salt thereof to a related-art hepatocyte differentiation-inducing medium. Thus, the inventors have completed the present invention.

That is, the present disclosure includes the following.

1. A production method for hepatocyte lineage cells, comprising culturing pluripotent stem cells in a hepatocyte differentiation-inducing medium containing lactic acid or a salt thereof.

2. A production method according to the above-mentioned item 1, wherein the lactic acid or the salt thereof comprises one of calcium lactate, sodium lactate, and lactic acid.

3. A production method according to the above-mentioned item 1 or 2, wherein the pluripotent stem cells comprise induced pluripotent stem (iPS) cells.

4. A production method according to the above-mentioned item 1 or 2, wherein the hepatocyte lineage cells comprise hepatoblasts.

5. A production method for hepatoblasts, comprising culturing iPS in a hepatocyte differentiation-inducing medium containing calcium lactate, sodium lactate, or lactic acid.

6. A production method according to the above-mentioned item 1 or 5, wherein a concentration of the lactic acid or the salt thereof in the medium is from 1 μM to 100 mM.

7. A production method according to the above-mentioned item 1 or 5, wherein the lactic acid or the salt thereof in the medium comprises 5 μM to 65 mM calcium lactate.

8. A production method according to the above-mentioned item 1 or 5, wherein the lactic acid or the salt thereof in the medium comprises 300 μM to 30 mM calcium lactate.

9. A production method according to the above-mentioned item 1 or 5, wherein the lactic acid or the salt thereof in the medium comprises 5 μM to 2 mM calcium lactate.

10. A production method according to the above-mentioned item 1 or 5, wherein the lactic acid or the salt thereof in the medium comprises 300 μM to 2 mM calcium lactate.

11. A production method according to the above-mentioned item 1 or 5, wherein the lactic acid or the salt thereof in the medium comprises 1 μM to 50 mM sodium lactate.

12. A production method according to the above-mentioned item 1 or 5, wherein the lactic acid or the salt thereof in the medium comprises 1 μM to 100 mM lactic acid.

13. A production method according to the above-mentioned item 1 or 5, wherein the medium contains pyruvic acid or a salt thereof.

14. A production method according to the above-mentioned item 12, wherein a pyruvic acid concentration in the medium is from 1 μM to 20 mM.

15. A hepatocyte lineage cell, which is produced by the method of the above-mentioned item 1 or 5.

16. A cell culture product, which is produced by the method of the above-mentioned item 1 or 5.

17. A production method for hepatocyte lineage cells, including culturing pluripotent stem cells in a hepatocyte differentiation-inducing medium containing lactic acid or a salt thereof.

18. A production method according to the above-mentioned item 17, wherein the lactic acid or the salt thereof includes one of calcium lactate, sodium lactate, and lactic acid.

19. A production method according to the above-mentioned item 17 or 18, wherein the pluripotent stem cells include induced pluripotent stem (iPS) cells.

20. A production method according to any one of the above-mentioned items 17 to 19, wherein the hepatocyte lineage cells include hepatoblasts.

21. A production method according to any one of the above-mentioned items 17 to 20, wherein a concentration of the lactic acid or the salt thereof in the medium is from 1 μM to 100 mM.

22. A production method according to any one of the above-mentioned items 17 to 21, wherein the lactic acid or the salt thereof in the medium includes 5 μM to 65 mM calcium lactate.

23. A production method according to any one of the above-mentioned items 17 to 21, wherein the lactic acid or the salt thereof in the medium includes 300 μM to 30 mM calcium lactate.

24. A production method according to any one of the above-mentioned items 17 to 21, wherein the lactic acid or the salt thereof in the medium includes 5 μM to 2 mM calcium lactate.

25. A production method according to any one of the above-mentioned items 17 to 21, wherein the lactic acid or the salt thereof in the medium includes 300 μM to 2 mM calcium lactate.

26. A production method according to any one of the above-mentioned items 17 to 21, wherein the lactic acid or the salt thereof in the medium includes 1 μM to 50 mM sodium lactate.

27. A production method according to any one of the above-mentioned items 17 to 21, wherein the lactic acid or the salt thereof in the medium includes 1 μM to 100 mM lactic acid.

28. A production method according to any one of the above-mentioned items 17 to 27, wherein the medium contains pyruvic acid or a salt thereof.

29. A production method according to the above-mentioned item 28, wherein a pyruvic acid concentration in the medium is from 1 μM to 20 mM.

30. A hepatocyte lineage cell, which is produced by the method of any one of the above-mentioned items 17 to 29.

31. A cell culture product, which is produced by the method of any one of the above-mentioned items 17 to 29.

32. A hepatocyte differentiation-inducing medium, including:

a composition shown in Table 1 below; and

lactic acid or a salt thereof at a lactic acid concentration of from 1 μM to 100 mM.

TABLE 1

| | | | |
|---|---|---|---|
| L15-ES medium | Inorganic salts | | |
| (1 L) | CaCl₂•2H₂O 1M | 1.258 | ml |
| | solution | | |
| | MgCl₂•6H₂O | 0.203 | g |
| | MgSO₄ (anhyd) | 0.098 | g |
| | KCl 1M solution | 5.3655 | ml |
| | KH₂PO₄ | 0.06 | g |
| | NaCl 5M solution | 27.29 | ml (7.915 g) |
| | NaH₂PO₄•2H₂O | 0.14014 | g |
| | Amino acids | | |
| | L-Alanine | 0.225 | g |
| | L-Arginine•HCl | 0 | g |
| | L-Asparagine•H₂O | 0.25 | g |
| | L-Cysteine | 0.12 | g |
| | L-Cystine•2HCl | 0 | g |
| | L-Glutamine | 0.3 | g |
| | Glycine | 0.2 | g |
| | L-Histidine•HCl•H₂O | 0.25 | g |
| | L-Isoleucine | 0.25 | g |
| | L-Leucine | 0.125 | g |
| | L-Lysine•HCl | 0.075 | g |
| | L-Methionine | 0.075 | g |
| | L-Phenylalanine | 0.125 | g |
| | L-Serine | 0.2 | g |
| | L-Threonine | 0.3 | g |
| | L-Tryptophan | 0.02 | g |
| | L-Tyrosine•2Na•H₂O | 0 | g |
| | L-Valine | 0.1 | g |
| | L-Ornithine•HCl | 0.169 | g |
| Gibco MEM | Vitamins | | |
| vitamins | Sodium chloride | 0.085 | g |
| (diluted | Choline chloride | 0.001 | g |
| 100-fold) 10 ml | Folic acid | 0.001 | g |
| | myo-Inositol | 0.001 | g |
| | Niacinamide | 0.001 | g |
| | D-Pantothenic | 0.001 | g |
| | acid•½Ca | | |
| | Pyridoxal•HCl | 0.001 | g |
| | Riboflavin | 0.0001 | g |
| | Thiamine•HCl | 0.001 | g |
| Others | Others | | |
| | D(+)-Galactose | 0.9 | g |
| | D-Glucose | 0 | g |
| | Phenol red•Na | 0.01 | g |
| | Glycerol (specific | 0.365 | ml |
| | gravity: 1.26 g/ml) | | |
| | Sodium pyruvate | 0 | ml |
| | Proline | 0.03 | g |
| | 7.5% Sodium | 36.6 | ml |
| | hydrogen carbonate | | |
| | solution | | |
| | Mercaptoethanol | 1,000 | μl |
| | (10⁻⁴M solution) | | |
| | Non-essential amino | 0 | ml |
| | acids | | |

33. A hepatocyte differentiation-inducing medium, including:

a composition shown in Table 2 below; and

lactic acid or a salt thereof at a lactic acid concentration of from 1 μM to 100 mM.

TABLE 2

| HDI (1 L) | Inorganic salts | | |
|---|---|---|---|
| | CaCl₂•2H₂O | 0.185 | g |
| | MgCl₂•6H₂O | 0.203 | g |
| | MgSO₄ (anhyd) | 0.098 | g |
| | KCl | 0.4 | g |
| | KH₂PO₄ | 0.06 | g |
| | NaCl | 7.915 | g |
| | Na₂HPO₄ | 0.19 | g |
| | Amino acids | | |
| | L-Alanine | 0.225 | g |
| | L-Asparagine•H₂O | 0.25 | g |
| | L-Cysteine | 0.12 | g |
| | Glycine | 0.2 | g |
| | L-Histidine•HCl•H₂O | 0.25 | g |
| | L-Isoleucine | 0.25 | g |
| | L-Leucine | 0.125 | g |
| | L-Lysine•HCl | 0.075 | g |
| | L-Methionine | 0.075 | g |
| | L-Phenylalanine | 0.125 | g |
| | L-Serine | 0.2 | g |
| | L-Threonine | 0.3 | g |
| | L-Tryptophan | 0.02 | g |
| | L-Valine | 0.1 | g |
| | Others | | |
| | Phenol red•Na | 0.01 | g |
| | NaHCO₃ | 2.745 | g |
| | MEM vitamin | 10 | ml |
| | solution | | |
| | (concentrated | | |
| | 100-fold) | | |
| | KnockOut Serum | 100 | ml |
| | Replacement | | |
| | Glutamine | 0.3 | g |
| | Ornithine | 0.169 | g |
| | Galactose | 0.9 | g |
| | Oncostatin M | 0.02 | g |
| | FPH1 | 3.88 | g |
| | M50054 | 100 | mg |
| | Non-essential amino | 10 | ml |
| | acids | | |
| | Sodium pyruvate | 10 | ml |
| | Nicotinamide | 1.2 | g |
| | Proline | 0.03 | g |

According to the present disclosure, the method of efficiently producing hepatocyte lineage cells from pluripotent stem cells, such as iPS cells, can be provided.

### BRIEF DESCRIPTION OF THE DRAWINGS

FIG. 1 includes graphs for showing, in a metabolic pathway, average values for each medium of the quantification results of typical metabolites in human metabolome analysis in Example 1. In FIG. 1, a bar on the left side of each of bar graphs represents a result for a control (ReproFF), and a bar on the right side represents a result for HDI.

FIG. 2A, FIG. 2B, and FIG. 2C are graphs for showing cell proliferation rates (%) in culture in HSM supplemented with lactic acid in Example 2. ReproFF serving as a pluripotency-maintaining medium was defined as 100%. FF in each of the figures represents a result for ReproFF, results for HSM supplemented with calcium lactate are shown in FIG. 2A, results for HSM supplemented with sodium lactate are shown in FIG. 2B, and results for HSM supplemented with lactic acid are shown in FIG. 2C.

FIG. 3A and FIG. 3B are photographs for showing the results of optical microscope observation in Example 3 (original magnification: ×200, scale bar: 50 mm.). A result for 3 mM sodium lactate is shown in FIG. 3A (the arrow indicates precipitates), and a result for 1 mM calcium lactate is shown in FIG. 3B (the arrowhead indicates viable cells).

FIG. 4A and FIG. 4B are graphs for showing the results of the expressions of AFP and albumin in Example 4. Results for AFP are shown in FIG. 4A, and results for albumin are shown in FIG. 4B.

FIG. 5A, FIG. 5B, and FIG. 5C are photographs for showing the results of optical microscope observation in Example 5 (original magnification: ×400, scale bar: 25 μm.). Cells cultured with calcium lactate are shown in FIG. 5A, cells cultured with sodium lactate are shown in FIG. 5B, and cells cultured with lactic acid are shown in FIG. 5C. The arrowhead in each of FIG. 5A, FIG. 5B, and FIG. 5C indicates viable cells.

FIG. 6A and FIG. 6B are photographs for showing the results of immunostaining in Example 6 (original magnification: ×400, scale bar: 50 mm.). The result of 7-day culture in HSM supplemented with lactic acid is shown in FIG. 6A, and the result of a negative control cultured in ReproFF for 7 days and having added thereto no anti-AFP antibody is shown in FIG. 6B.

FIG. 7 is a graph for showing the results of culture in HSM supplemented with pyruvic acid in Example 7. In FIG. 7, P represents HSM medium supplemented with pyruvic acid.

### DESCRIPTION OF THE EMBODIMENTS

The present disclosure relates to a production method for hepatocyte lineage cells, a hepatocyte lineage cell or culture product obtained by the production method, and a hepatocyte differentiation-inducing medium. The details thereof are described below.

(Pluripotent Stem Cells)

In the present disclosure, any suitable pluripotent stem cells may be used. The pluripotent stem cells refer to stem cells having a self-renewal ability for a long period of time under predetermined culture conditions, and having pluripotency to many kinds of cells under predetermined differentiation-inducing conditions. Any cells may be used as the pluripotent stem cells as long as the cells have both a self-renewal ability to renew themselves and pluripotency. Specific examples thereof may include iPS cells and ES cells. Of those, induced pluripotent stem cells are preferably recommended.

The pluripotent stem cells maybe derived from vertebrate cells. The pluripotent stem cells may be derived from mammalian cells, such as rodent cells, mouse cells, primate cells, or human cells.

(Induced Pluripotent Stem Cells)

The induced pluripotent stem cells (iPS cells) are pluripotent stem cells to be induced from somatic cells, such as human fibroblasts, by a genetic recombination technology. The iPS cells may be derived from any species as long as the cells are produced from somatic cells of mammals, such as humans and mice. However, when the iPS cells are used in regenerative medicine, such as transplantation, ones produced from somatic cells derived from a species serving as a target of the regenerative medicine are preferred, and ones produced from somatic cells collected from an individual of the target are more preferred.

The iPS cells may be prepared by any method to be generally used. In addition, the iPS cells may be passaged by using a known method involving maintaining and culturing the cells in an undifferentiated state.

Examples of the pluripotent stem cells may include iPS cells, and 201B7 cells, which are iPS cells.

(Hepatocyte Lineage Cells)

The hepatocyte lineage cells of the present disclosure mean cells at all stages of a process in which cells determined to differentiate from pluripotent stem cells to hepatocytes differentiate to mature hepatocytes via immature hepatocytes. The differentiation may be partial or full. Examples thereof may include hepatocytes, hepatic progenitor cells, mature hepatocytes, and hepatoblasts.

The hepatocyte lineage cells may preferably express at least one hepatocyte-specific cell surface marker. The hepatocyte lineage cells may more preferably express α-fetoprotein (AFP), albumin, or both thereof. In addition, the hepatocyte lineage cells may be immortal cells. In addition, intermediate cells mean cells in the process of differentiation of pluripotent stem cells to mature hepatocytes.

(Hepatocytes)

The hepatocytes as used herein are meant to include cells at all differentiation stages determined to differentiate to hepatocytes, such as hepatic progenitor cells and mature hepatocytes.

There are a report that the hepatic progenitor cells are cells having an ability to actively proliferate and differentiate to hepatocytes and bile duct epithelium, which are found at the fetal stage (Kakinuma, S. et al., “Analyses of cell surface molecules on hepatic stem/progenitor cells in mouse fetal liver,” Journal of Hepatology, 2009, Vol. 51, No. 1, p. 127-138) and a report that the hepatic progenitor cells are small and oval cells to be generated in a liver regeneration process (Sangan, C. B. et al., “Hepatic progenitor cells,” Cell and Tissue Research, 2010, Vol. 342, No. 2, p. 131-137). Thus, the hepatic progenitor cells have a proliferation ability and an ability to differentiate to hepatocytes and bile duct epithelial cells. The hepatic progenitor cells have a higher proliferation ability than the mature hepatocytes, and also form bile duct epithelium. Therefore, the transplantation of the hepatic progenitor cells to liver rapidly forms the existing liver construction and can be expected to regenerate lost liver more effectively than the transplantation of only hepatocytes.

The mature hepatocytes are also called mature hepatic parenchymal cells, and are terminally differentiated cells expressing a wide variety of liver-specific functions, for example, functions such as a cholesterol synthesis ability, amino acid transport activity, and glucose-6-phosphatase activity. Meanwhile, the mature hepatocytes have an active proliferation ability as well known in a liver regeneration phenomenon.

(Hepatoblasts)

The hepatoblasts are foregut endoderm-derived tissue stem cells, and are cells essential for liver tissue development. The hepatoblasts are supposed to be tissue stem cells in fetal liver, and are present in an extremely small number in mature liver. The hepatoblasts present in mature liver are considered to be activated in association with liver damage and play an important role in liver repair. It has been reported that many transcription factors, and various extracellular matrices to be produced by nonparenchymal cells, such as hepatocyte growth factor (HGF) and transforming growth factor β (TGF-β), are involved in the differentiation of hepatoblasts to hepatocytes. The hepatoblasts may be cultured in vitro, and may be cultured under appropriate culture conditions so as to differentiate to hepatic parenchymal cells or bile duct epithelial cells.

The hepatoblasts may express α-fetoprotein (AFP), albumin, or both thereof.

(Hepatocyte Differentiation-Inducing Medium)

A hepatocyte differentiation-inducing medium of the present disclosure is not particularly limited as long as the medium contains lactic acid and can differentiate pluripotent stem cells to hepatocyte lineage cells, and may be produced by adding lactic acid or a salt thereof to a hepatocyte differentiation-inducing medium known per se.

For example, the following may be given as an example.

The hepatocyte differentiation-inducing medium of the present disclosure may be produced by adding lactic acid or a salt thereof to hepatocyte selection medium (HSM) (Tomizawa, M. et al., “Survival of primary human hepatocytes and death of induced pluripotent stem cells in media lacking glucose and arginine,” PLoS One, 2013, Vol. 8, e71897) or hepatocyte differentiation inducer (HDI) (Tomizawa, M. et al., “An optimal medium supplementation regimen for initiation of hepatocyte differentiation inhuman induced pluripotent stem cells,” Journal of Cellular Biochemistry, 2015, Vol. 116, No. 8, p. 1479-1489).

HDI stands for hepatocyte differentiation inducer, and is a culture medium prepared by adding oncostatin M, hepatocyte functional proliferation inducer 1 (FPH1), M50054, non-essential amino acids, sodium pyruvate, nicotinamide, and L-glutamine to HSM. Although HSM contains L-glutamine, L-glutamine is rapidly decomposed, and hence L-glutamine is added at the time of the preparation of HDI.

HSM may be specifically exemplified by a medium having a composition shown in Table 3 below.

TABLE 3

| | | | |
|---|---|---|---|
| L15-ES medium | Inorganic salts | | |
| (1 L) | CaCl₂•2H₂O 1M solution | 1.258 | ml |
| | MgCl₂•6H₂O | 0.203 | g |
| | MgSO₄ (anhyd) | 0.098 | g |
| | KCl 1M solution | 5.3655 | ml |
| | KH₂PO₄ | 0.06 | g |
| | NaCl 5M solution | 27.29 | ml (7.915 g) |
| | NaH₂PO₄•2H₂O | 0.14014 | g |
| | Amino acids | | |
| | L-Alanine | 0.225 | g |
| | L-Arginine•HCl | 0 | g |
| | L-Asparagine•H₂O | 0.25 | g |
| | L-Cysteine | 0.12 | g |
| | L-Cystine•2HCl | 0 | g |
| | L-Glutamine | 0.3 | g |
| | Glycine | 0.2 | g |
| | L-Histidine•HCl•H₂O | 0.25 | g |
| | L-Isoleucine | 0.25 | g |
| | L-Leucine | 0.125 | g |
| | L-Lysine•HCl | 0.075 | g |
| | L-Methionine | 0.075 | g |
| | L-Phenylalanine | 0.125 | g |
| | L-Serine | 0.2 | g |
| | L-Threonine | 0.3 | g |
| | L-Tryptophan | 0.02 | g |
| | L-Tyrosine•2Na•H₂O | 0 | g |
| | L-Valine | 0.1 | g |
| | L-Ornithine•HCl | 0.169 | g |
| Gibco MEM | Vitamins | | |
| vitamins | Sodium chloride | 0.085 | g |
| (diluted | Choline chloride | 0.001 | g |
| 100-fold) 10 ml | Folic acid | 0.001 | g |
| | myo-Inositol | 0.001 | g |
| | Niacinamide | 0.001 | g |
| | D-Pantothenic | 0.001 | g |
| | acid•½Ca | | |
| | Pyridoxal•HCl | 0.001 | g |
| | Riboflavin | 0.0001 | g |
| | Thiamine•HCl | 0.001 | g |
| Others | Others | | |
| | D(+)-Galactose | 0.9 | g |
| | D-Glucose | 0 | g |
| | Phenol red•Na | 0.01 | g |
| | Glycerol (specific | 0.365 | ml |
| | gravity: 1.26 g/ml) | | |
| | Sodium pyruvate | 0 | ml |
| | Proline | 0.03 | g |
| | 7.5% Sodium hydrogen | 36.6 | ml |
| | carbonate solution | | |
| | Mercaptoethanol (10⁻⁴M | 1,000 | μl |
| | solution) | | |
| | Non-essential amino | 0 | ml |
| | acids | | |

HDI is preferably supplemented with serum or a serum replacement, preferably a serum replacement before use. Any serum may be used as long as the serum is generally used for the culture of pluripotent stem cells, hepatoblasts, and hepatocytes. When the serum is used, it is preferred to use serum derived from a living organism of the same species as the species of cells to be cultured. For example, when the cells to be cultured are human cells, it is preferred to use human-derived serum. The serum replacement is a substance to be used for the maintenance and growth of cells in place of the serum, and means a composition having a known chemical composition. Any serum replacement may be used as long as the serum replacement is generally used for the culture of pluripotent stem cells, hepatoblasts, and hepatocytes. Examples thereof may include Knockout™ Serum Replacement (manufactured by Life Technologies), CDM-HD Serum Replacement (manufactured by FiberCell Systems), StemSure Serum Replacement (manufactured by Wako Pure Chemical Industries, Ltd.), and Nu-Serum™ (manufactured by Becton Dickinson). The dose of the serum or the serum replacement may be determined by simple repeated experiments.

HDI may be specifically exemplified by a medium having a composition shown in Table 4 below. HDI may be a medium having a composition in which insulin, dexamethasone, and aprotinin are added to the composition shown in Table 3. The presence or absence of the addition of insulin, dexamethasone, and aprotinin to a medium has substantially no influence on an inducing effect on differentiation of iPS cells to hepatoblasts. Insulin may be added so that its final concentration is from 10⁻⁸ M to 10⁻¹⁰ M, preferably from 10⁻⁹ M to 10⁻¹⁰ M, more preferably 10⁻⁹M. Dexamethasone may be added so that its final concentration is from 10⁻⁸ M to 10⁻¹⁰ M, preferably from 10⁻⁹ M to 10⁻¹⁰ M, more preferably 10⁻⁹M. Aprotinin may be added so that its final concentration is from 10 U/ml to 300 U/ml, preferably from 30 U/ml to 200 U/ml, more preferably from 50 U/ml to 100 U/ml, still more preferably 50 U/ml. The addition concentrations of insulin, dexamethasone, and aprotinin are not limited to those exemplified concentrations, and may each be any concentration as long as the differentiation of induced pluripotent stem cells to hepatoblasts can be induced. The addition concentrations may each be easily determined by simple repeated experiments.

TABLE 4

| HDI (1 L) | Inorganic salts | | |
|---|---|---|---|
| | CaCl₂•2H₂O | 0.185 | g |
| | MgCl₂•6H₂O | 0.203 | g |
| | MgSO₄ (anhyd) | 0.098 | g |
| | KCl | 0.4 | g |
| | KH₂PO₄ | 0.06 | g |
| | NaCl | 7.915 | g |
| | Na₂HPO₄ | 0.19 | g |
| | Amino acids | | |
| | L-Alanine | 0.225 | g |
| | L-Asparagine•H₂O | 0.25 | g |
| | L-Cysteine | 0.12 | g |
| | Glycine | 0.2 | g |
| | L-Histidine•HCl•H₂O | 0.25 | g |
| | L-Isoleucine | 0.25 | g |
| | L-Leucine | 0.125 | g |
| | L-Lysine•HCl | 0.075 | g |
| | L-Methionine | 0.075 | g |
| | L-Phenylalanine | 0.125 | g |
| | L-Serine | 0.2 | g |
| | L-Threonine | 0.3 | g |
| | L-Tryptophan | 0.02 | g |
| | L-Valine | 0.1 | g |
| | Others | | |
| | Phenol red•Na | 0.01 | g |
| | NaHCO₃ | 2.745 | g |
| | MEM vitamin | 10 | ml |
| | solution | | |
| | (concentrated | | |
| | 100-fold) | | |
| | KnockOut Serum | 100 | ml |
| | Replacement | | |
| | Glutamine | 0.3 | g |
| | Ornithine | 0.169 | g |
| | Galactose | 0.9 | g |
| | Oncostatin M | 0.02 | g |
| | FPH1 | 3.88 | g |
| | M50054 | 100 | mg |
| | Non-essential amino | 10 | ml |
| | acids | | |
| | Sodium pyruvate | 10 | ml |
| | Nicotinamide | 1.2 | g |
| | Proline | 0.03 | g |

In the medium having a composition shown in Table 4, the concentrations of constituents of MEM vitamin solution in terms of final concentration are as follows: 0.085 g of sodium chloride; 0.001 g of choline chloride; 0.001 g of folic acid; 0.001 g of myo-inositol; 0.001 g of niacinamide; 0.001 g of D-pantothenic acid.½Ca; 0.001 g of pyridoxal.HCl; 0.0001 g of riboflavin; and 0.001 g of thiamine.HCl.

In addition, the method according to the present disclosure may be a method involving preculturing pluripotent stem cells in a culture medium other than HSM and HDI, then changing the culture medium to HSM and/or HDI, and further culturing the cells for at least 2 days or more, preferably from 2 days to 7 days, still more preferably from 2 days to 4 days, yet still more preferably 2 days. Examples of the culture medium to be used in the preculture may include Leibovitz's-15 medium, William's E medium, and Dulbecco's Modified Eagle Medium: Nutrient Mixture F-12. A more preferred example thereof may be Leibovitz's-15 medium.

Those culture media are each preferably supplemented with serum or a serum replacement, preferably a serum replacement before use. In addition, those culture media may each be supplemented with an effective amount of a compound or a reagent having an effect on the growth of desired cells, such as proline or nicotinamide, before use. A period of time for the preculture is preferably at least 2 days or more, more preferably 7 days. As culture conditions for the preculture, there may be used any of general culture conditions, preferably known conditions that have been used in the culture of pluripotent stem cells and in culture for inducing differentiation of pluripotent stem cells to hepatoblasts or hepatocytes. Specific examples thereof may include culture conditions to be performed at from 35° C. to 40° C., preferably 37° C. under the atmosphere of 95% air and 5% CO₂. A cell survival rate is increased in the case where the pluripotent stem cells are precultured in a culture medium other than HSM and HDI and then cultured in HSM and/or HDI as compared to the case where the pluripotent stem cells are not precultured, which may result in an increase in number of hepatoblasts to be obtained.

(Lactic Acid or Salt Thereof)

The lactic acid or the salt thereof that may be used in the present disclosure is not particularly limited, and examples thereof include calcium lactate, sodium lactate, lactic acid, and potassium lactate.

The concentration of the lactic acid or the salt thereof in the hepatocyte differentiation-inducing medium of the present disclosure is not particularly limited as long as the pluripotent stem cells can be differentiated to hepatocyte lineage cells. The concentration may be from 1 μM to 1,000 mM, and is preferably from 3 μM to 50 mM, more preferably from 10 μM to 30 mM, still more preferably from 100 μM to 1 mM.

More specifically, in consideration of the results of Examples, when calcium lactate is added, its concentration falls within the range of from 5 μM to 65 mM, preferably the range of from 10 μM to 30 mM, more preferably the range of from 300 μM to 30 mM. When sodium lactate is added, its concentration falls within the range of from 1 μM to 50 mM, preferably the range of from 3 μM to 30 mM. When lactic acid is added, its concentration falls within the range of from 1 μM to 100 mM, preferably the range of from 3 μM to 100 mM.

In addition, in consideration of the suppression of precipitate formation, the concentration of the lactic acid or the salt thereof in the hepatocyte differentiation-inducing medium preferably falls within the range of 2 mM or less, particularly 1 mM or less. That is, when calcium lactate is added, its concentration falls within range of from 5 μM to 2 mM (or from 5 μM to 1 mM), preferably the range of from 10 μM to 2 mM (or from 10 μM to 1 mM), more preferably the range of from 300 μM to 2 mM (or from 300 μM to 1 mM). When sodium lactate is added, its concentration falls within the range of from 1 μM to 2 mM (or from 1 μM to 1 mM), preferably the range of from 3 μM to 2 mM (or from 3 μM to 1 mM). When lactic acid is added, its concentration falls within the range of from 1 μM to 2 mM (or from 1 μM to 1 mM), preferably the range of from 3 μM to 2 mM (or from 3 μM to 1 mM).

Further, in consideration of the results of Example 5 and Example 6, the lactic acid or the salt thereof in the hepatocyte differentiation-inducing medium of the present disclosure is preferably calcium lactate.

In consideration of the foregoing, the most preferred conditions are as follows: the hepatocyte differentiation-inducing medium contains calcium lactate in the range of from 300 μM to 2 mM or from 300 μM to 1 mM.

(Pyruvic Acid or Salt Thereof)

The pyruvic acid or the salt thereof that may be used in the present disclosure is not particularly limited, and examples thereof include sodium pyruvate and pyruvic acid. Of those, sodium pyruvate is particularly preferred.

The concentration of the pyruvic acid or the salt thereof in the hepatocyte differentiation-inducing medium of the present disclosure is not particularly limited. The concentration may be from 1 μM to 20 mM, and is preferably from 3 μM to 10 mM.

(Culture)

Culture conditions of the present disclosure are not particularly limited as long as the pluripotent stem cells can be differentiated to hepatocyte lineage cells, and culture may be performed for any appropriate period of time. For example, the culture of the cells may be performed for at least 5 minutes, at least 15 minutes, at least 30 minutes, at least 45 minutes, at least 1 hour, at least 2 hours, at least 3 hours, at least 5 hours, at least 12 hours, at least 1 day, at least 3 days, at least 7 days (1 week), at least 8 days, at least 10 days, at least 12 days, at least 15 days, at least 20 days, at least 28 days, at least 1 month, at least 6 weeks, at least 2 months, at least 3 months, at least 6 months, at least 9 months, at least 1 year, 5 minutes or less, 1 year or more, or any period therebetween. The culture of the cells may be performed for, for example, from about 4 days to about 12 days. The culture of the cells may be performed for, for example, at least 7 days or 7 days or more, but is performed for preferably at least 3 days or more, more preferably from 3 days to 12 days, still more preferably from 3 days to 7 days, particularly preferably 7 days.

The culture period may mean the total period for which the cells are cultured, or a part thereof or a stretch of time therein. For example, the period may mean that the cells are cultured in the hepatocyte differentiation-inducing medium in a period before the completion of differentiation or a period after the differentiation. The cells may be cultured until the expression of at least one hepatocyte lineage cell-specific marker exceeds a threshold for recognition. The marker may be one associated with immature hepatocytes, mature hepatocytes, or both thereof. The cells may be cultured until the expression of a marker of the original cells falls below a threshold for recognition. The cells may be cultured until the cells have the morphology of hepatocyte lineage cells and/or hepatocytes.

The culture of the present disclosure may be performed in vivo, in vitro, or both in vivo and in vitro. A culture environment may include a liquid medium, a solid support, or both thereof. For example, the culture environment may contribute to the formation of a liver tissue, a part of liver, or a whole liver. The differentiated cells may be transplanted to a mammalian recipient, for example, a rodent, such as a mouse, or a primate, such as a human.

(Hepatocyte Lineage Cells Obtained by Method of the Present Disclosure)

Hepatocyte lineage cells acquired by the method of the present disclosure may be immortal cells, or may have a limited number of replication cycles. The hepatocyte lineage cells may be in the form of cells in cell culture in a liquid, solid, or liquid/solid support. The hepatocyte lineage cells may be in the form of a tissue or organ suitable for transplantation to an organism, such as liver. The hepatocyte lineage cells may more preferably express α-fetoprotein, albumin, or both thereof.

(Cell Culture Product Obtained by Method of the Present Disclosure)

The cell culture product obtained by the method of the present disclosure is a cell culture product containing hepatocyte lineage cells, particularly hepatoblasts. The “cell culture product” refers to a cell group obtained after culturing cells. The cell culture product of the present disclosure is substantially free of cells having pluripotency. The “substantially free” refers to that the ratio of the number of hepatoblasts to the number of cells having pluripotency (the number of hepatoblasts: the number of cells having pluripotency) is 1,000:1 or less, preferably 10,000:1 or less, more preferably 100,000:1 or less. In addition, the cell culture product of the present disclosure may contain, in addition to the hepatoblasts, cells further differentiated from the hepatoblasts.

Further, the cell culture product obtained by the method of the present disclosure may be used as, for example, a drug in regenerative medicine including transplantation treatment for a liver disease, such as fulminant hepatitis, or liver failure occurring after partial hepatectomy or in the natural course of liver cirrhosis. A drug containing as an active ingredient the cell culture product of the present disclosure may contain physiological saline, an additive, a medium, or the like which is pharmacologically acceptable, and is preferably not contaminated with impurities, such as foreign serum and a virus.

The cell culture product obtained by the method of the present disclosure can be differentiated to hepatic parenchymal cells and bile duct epithelial cells by in vitro culture under appropriate culture conditions. For example, it has been reported that many transcription factors, and various extracellular matrices to be produced by non-parenchymal cells, such as HGF and TGF-β, are involved in the differentiation of hepatoblasts to hepatocytes. The induction of differentiation of hepatoblasts to hepatocytes may be carried out in vitro through the utilization of those substances.

(Reagent and Reagent Kit for Production of Hepatocyte Lineage Cells from Pluripotent Stem Cells)

The present disclosure relates to a reagent and reagent kit for production of hepatocyte lineage cells from pluripotent stem cells. The reagent and the reagent kit each include at least lactic acid or a salt thereof, and as required, pyruvic acid or a salt thereof.

In addition, the reagent and the reagent kit of the present disclosure may be exemplified by: such a medium that the lactic acid or the salt thereof is contained in a hepatocyte differentiation-inducing medium known per se so as to have a lactic acid concentration of from 3 μM to 100 mM; such a medium that the lactic acid or the salt thereof is contained in a medium containing a composition shown in Table 3 so as to have a lactic acid concentration of from 3 μM to 100 mM; or such a medium that the lactic acid or the salt thereof is contained in a medium containing a composition shown in Table 4 so as to have a lactic acid concentration of from 3 μM to 100 mM.

(Confirmation Method for Differentiation Induction)

The induction of differentiation of pluripotent stem cells to hepatocyte lineage cells by the present disclosure may be confirmed by detecting the expression of a known marker of hepatoblasts or hepatocytes. Examples of such marker may include AFP and DLK-1, markers of hepatoblasts, and enzymes characteristically expressed in hepatocytes, such as CYP3A4, which is involved in drug metabolism, or ALDH2, which is involved in alcohol metabolism. In addition, an example of the marker of hepatocytes may be albumin.

The detection of the marker of hepatoblasts or hepatocytes may be performed by the reverse transcription of mRNA of a target protein in the cells, followed by measurement by a known genetic engineering technique, such as a polymerase chain reaction (PCR), a reverse transcriptase polymerase chain reaction (RT-PCR), or a real-time quantitative polymerase chain reaction, or may also be performed by confirmation by enzyme-linked immunoassay (ELISA method) or an immunostaining method using an antibody against a test protein. However, the detection is not limited to those methods, and any of known methods may be used.

The expression of Nanog, which is specifically expressed in pluripotent stem cells and early embryos, almost disappears in the culture cells obtained by the method according to the present disclosure, suggesting that the cells have lost pluripotency. Accordingly, such culture cells are considered to have an extremely low risk of forming a tumor when used in transplantation for liver disease treatment.

### EXAMPLES

The present disclosure is described in detail below by way of specific examples. However, the present disclosure is not limited to the examples.

### Example 1

(Human Metabolome Analysis)

Metabolome analysis was performed in order to analyze a change in metabolism in cells occurring after culture of iPS cells in HDI medium, which was a hepatocyte differentiation-inducing medium previously developed by the inventors of the present invention.

(Materials and Methods)

### 1. Preparation of Cells, Media, etc.

201B7 cells (obtained from RIKEN BioResource Research Center (BRC)) were used as the iPS cells.

Hepatocyte selection medium (HSM) was prepared to have a composition shown in Table 5 below. Hepatocyte differentiation inducer (HDI) was prepared to have a composition shown in Table 6 below.

A pluripotency-maintaining medium (ReproFF) was obtained from ReproCELL Inc.

A cell wash solution was prepared by adding 3 g of mannitol to Milli-Q water to a total volume of 60 ml.

An internal standard solution was prepared by adding 40 mL of an undiluted internal standard solution (provided by Human Metabolome Technologies, Inc.) to Milli-Q water to a total volume of 50 mL.

### 2. Culture

In 10 cm culture dishes coated with Matrigel, the 201B7 cells were cultured for 48 hours using 3 ml of HDI in each of three culture dishes, and 10 ml of ReproFF in one culture dish, to be brought into a 100% confluent state (1.0×10⁷ cells per 10 cm culture dish for ReproFF, and 2.1×10⁶ cells per 10 cm culture dish for HDI).

### 3. Preparation of Sample for Metabolome Analysis

After the 48-hour culture, the cell culture liquid was removed by aspiration, and the culture dish surface was washed with 10 ml of the cell wash solution. After the removal of the cell wash solution, washing was performed again with 2 ml of the cell wash solution.

After complete aspiration of the cell wash solution, 800 μL of methanol was added to each of the culture dishes, and shaking was repeated so that methanol covered the entire culture dish surface.

After the completion of the shaking, the culture dishes were left to stand for 30 seconds. Then, 550 μL of the internal standard solution was added, and shaking was repeated so that the mixed liquid covered the entire surface.

After the completion of the shaking, the culture dishes were left to stand for 30 seconds. Then, 1,000 μL of the mixed liquid was transferred to a 1.5 mL Eppendorf tube, which was left to stand on ice until the next operation.

The extract liquid (aqueous layer of two-layer separation) was collected and centrifuged (2,300×g, 4° C., 5 minutes).

350 μL each of the supernatant was transferred to filter cups of two ultrafiltration units (provided by Human Metabolome Technologies, Inc.).

The ultrafiltration units were centrifuged (9,100×g, 4° C., 2.5 hours) until there was no liquid in the filter cups.

The filter cups were removed from the samples after the centrifugation, lids were tightly shut, and then the samples were stored at −80° C. or less until sent to Human Metabolome Technologies, Inc.

### 4. Metabolome Analysis

For the samples prepared as described above, i.e., prepared by culturing the iPS cells in HDI medium or ReproFF serving as a pluripotency-maintaining medium, changes in metabolism in the cells were compared through metabolome analysis. The metabolome analysis was performed on contact by Human Metabolome Technologies, Inc.

TABLE 5

| | | | |
|---|---|---|---|
| L15-ES | Inorganic salts | | |
| medium (1 L) | CaCl₂•2H₂O 1M solution | 1.258 | ml |
| | MgCl₂•6H₂O | 0.203 | g |
| | MgSO₄ (anhyd) | 0.098 | g |
| | KCl 1M solution | 5.3655 | ml |
| | KH₂PO₄ | 0.06 | g |
| | NaCl 5M solution | 27.29 | ml (7.915 g) |
| | NaH₂PO₄•2H₂O | 0.14014 | g |
| | Amino acids | | |
| | L-Alanine | 0.225 | g |
| | L-Arginine•HCl | 0 | g |
| | L-Asparagine•H₂O | 0.25 | g |
| | L-Cysteine | 0.12 | g |
| | L-Cystine•2HCl | 0 | g |
| | L-Glutamine | 0.3 | g |
| | Glycine | 0.2 | g |
| | L-Histidine•HCl•H₂O | 0.25 | g |
| | L-Isoleucine | 0.25 | g |
| | L-Leucine | 0.125 | g |
| | L-Lysine•HCl | 0.075 | g |
| | L-Methionine | 0.075 | g |
| | L-Phenylalanine | 0.125 | g |
| | L-Serine | 0.2 | g |
| | L-Threonine | 0.3 | g |
| | L-Tryptophan | 0.02 | g |
| | L-Tyrosine•2Na•H₂O | 0 | g |
| | L-Valine | 0.1 | g |
| | L-Ornithine•HCl | 0.169 | g |
| Gibco MEM | Vitamins | | |
| vitamins | Sodium chloride | 0.085 | g |
| (diluted | Choline chloride | 0.001 | g |
| 100-fold) 10 ml | Folic acid | 0.001 | g |
| | myo-Inositol | 0.001 | g |
| | Niacinamide | 0.001 | g |
| | D-Pantothenic | 0.001 | g |
| | acid•½Ca | | |
| | Pyridoxal•HCl | 0.001 | g |
| | Riboflavin | 0.0001 | g |
| | Thiamine•HCl | 0.001 | g |
| Others | Others | | |
| | D(+)-Galactose | 0.9 | g |
| | D-Glucose | 0 | g |
| | Phenol red•Na | 0.01 | g |
| | Glycerol (specific | 0.365 | ml |
| | gravity: 1.26 g/ml) | | |
| | Sodium pyruvate | 0 | ml |
| | Proline | 0.03 | g |
| | 7.5% Sodium hydrogen | 36.6 | ml |
| | carbonate solution | | |
| | Mercaptoethanol (10⁻⁴M | 1,000 | μl |
| | solution) | | |
| | Non-essential amino | 0 | ml |
| | acids | | |

TABLE 6

| HDI (1 L) | Inorganic salts | | |
|---|---|---|---|
| | CaCl₂•2H₂O | 0.185 | g |
| | MgCl₂•6H₂O | 0.203 | g |
| | MgSO₄ (anhyd) | 0.098 | g |
| | KCl | 0.4 | g |
| | KH₂PO₄ | 0.06 | g |
| | NaCl | 7.915 | g |
| | Na₂HPO₄ | 0.19 | g |
| | Amino acids | | |
| | L-Alanine | 0.225 | g |
| | L-Asparagine•H₂O | 0.25 | g |
| | L-Cysteine | 0.12 | g |
| | Glycine | 0.2 | g |
| | L-Histidine•HCl•H₂O | 0.25 | g |
| | L-Isoleucine | 0.25 | g |
| | L-Leucine | 0.125 | g |
| | L-Lysine•HCl | 0.075 | g |
| | L-Methionine | 0.075 | g |
| | L-Phenylalanine | 0.125 | g |
| | L-Serine | 0.2 | g |
| | L-Threonine | 0.3 | g |
| | L-Tryptophan | 0.02 | g |
| | L-Valine | 0.1 | g |
| | Others | | |
| | Phenol red•Na | 0.01 | g |
| | NaHCO₃ | 2.745 | g |
| | MEM vitamin | 10 | ml |
| | solution | | |
| | (concentrated | | |
| | 100-fold) | | |
| | KnockOut Serum | 100 | ml |
| | Replacement | | |
| | Glutamine | 0.3 | g |
| | Ornithine | 0.169 | g |
| | Galactose | 0.9 | g |
| | Oncostatin M | 0.02 | g |
| | FPH1 | 3.88 | g |
| | M50054 | 100 | mg |
| | Non-essential amino | 10 | ml |
| | acids | | |
| | Sodium pyruvate | 10 | ml |
| | Nicotinamide | 1.2 | g |
| | Proline | 0.03 | g |

(Results)

The results of quantification are shown in Table 7. In Table 7, “3846FF-1” to “3846FF-3” represent the results of the cells cultured in ReproFF, and “3846HDI-1” to “3846HDI-3” represent the results of the cells cultured in HDI.

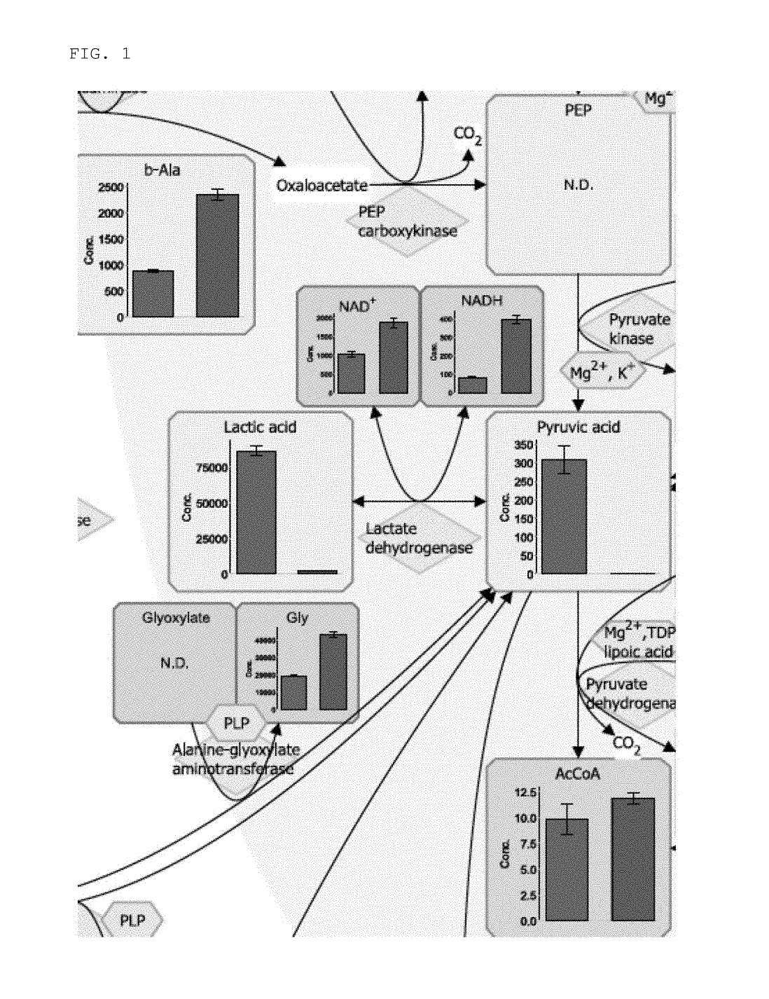

FIG. 1 includes graphs for showing, in a metabolic pathway, average values for each medium of the quantification results of typical metabolites. The metabolic pathway of FIG. 1 is an excerpt including the glycolysis-pyruvic acid-citric acid cycle and lactic acid portions.

As apparent from Table 7 and FIG. 1, for lactic acid, the group cultured in HDI had a decrease to 1/40 as compared to the control (ReproFF).

As apparent from FIG. 1, for pyruvic acid, the group cultured in HDI had a decrease as compared to the control (ReproFF).

The inventors of the present invention had assumed that, because HDI did not contain glucose, energy was produced through glycolysis of galactose. Galactose is converted to galactose-1-phosphate to enter glycolysis.

The results of this Example revealed that culture in HDI increased the amount of galactose-1-phosphate 1,000-fold. Thus, it was proved that galactose was, as expected, converted to galactose-1-phosphate to be utilized in energy production through glycolysis.

In addition, the metabolome analysis revealed that lactic acid was decreased to 1/40. Lactic acid is the end product of glycolysis. Therefore, although galactose was utilized in glycolysis, it was considered that the energy was insufficient for cell survival. That is, it is considered that, when pluripotent stem cells are cultured in HDI, the production of lactic acid is insufficient.

TABLE 7
Concentration (pml/10⁶ cells)

| | | | | | | | Control | | HDI | |
|---|---|---|---|---|---|---|---|---|---|---|
| | 3846FF-1 | 3846FF-2 | 3846FF-3 | 3846HDI-1 | 3846HDI-2 | 3846HDI-3 | Mean | SD | Mean | SD |
| Lactate | 89,699 | 83,510 | 86,074 | 2,393 | 1,841 | N. D. | 86,428 | 3,110 | 2,117 | 391 |
| Pyruvate | 344 | 306 | 270 | ND | ND | ND | 307 | 37 | ND | ND |

### Example 2

(Culture in HSM Supplemented with Lactic Acid)

It was confirmed from the results of Example 1 that, when pluripotent stem cells were cultured in HDI, the production of lactic acid was insufficient. HDI is obtained by adding agents and the like to HSM. In order to remove the influences of the agents added in HDI, iPS cells were cultured in HSM supplemented with lactic acid, and the efficiency of induction of hepatocytes was investigated.

(Materials and Methods)

### 1. Preparation of Cells, Media, etc.

201B7 cells were used as the iPS cells in the same manner as in Example 1.

HSM medium was prepared in the same manner as in Example 1.

Calcium lactate (manufactured by Wako Pure Chemical Industries, Ltd.), sodium lactate (manufactured by Wako Pure Chemical Industries, Ltd.), or lactic acid (manufactured by Kozakai Pharmaceutical Co., Ltd.) was used as a lactic acid source.

### 2. Culture

5×10⁴ 201B7 cells were seeded to a 96-well plate coated with Matrigel. The next day, to HSM, calcium lactate, sodium lactate, and lactic acid were each added at 3 μM, 10 μM, 30 μM, 100 μM, 300 μM, 1 mM, 3 mM, 10 mM, 30 mM, or 100 mM, followed by culture for 3 days. Control cells were cultured in ReproFF or HSM.

### 3. MTS Assay

After 3 days from the initiation of the culture, MTS assay (CellTiter 96 (registered trademark in Japan) AQueous One Solution, manufactured by Promega) was performed to investigate cell proliferation.

(Results)

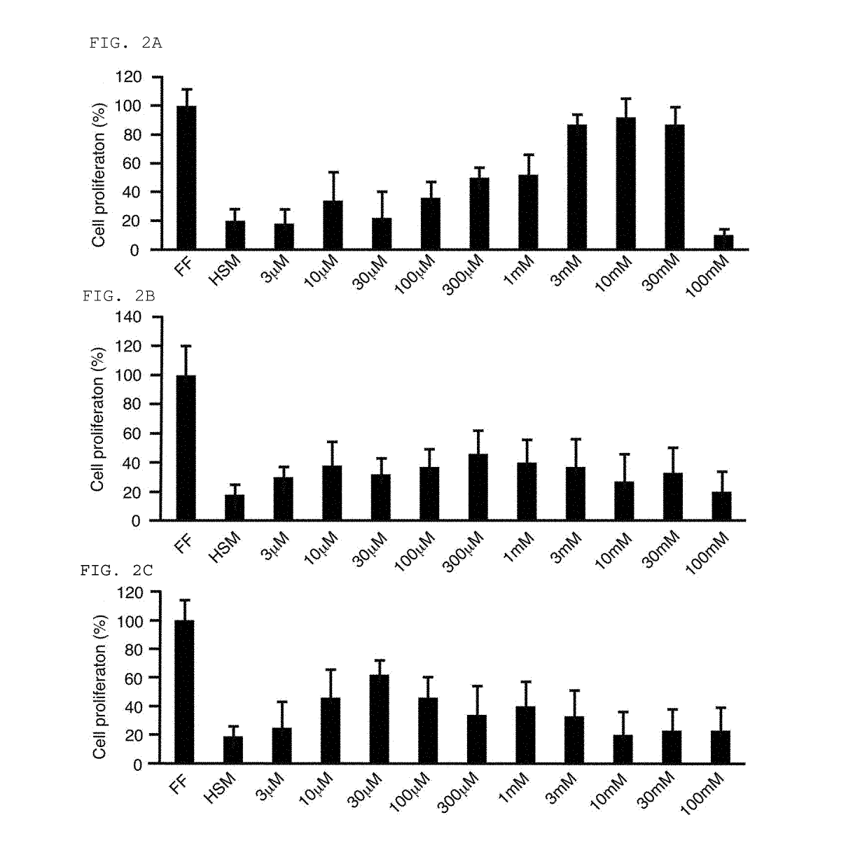

The results of the cell proliferation are shown in FIG. 2A, FIG. 2B, and FIG. 2C.

HSM supplemented with calcium lactate was confirmed to have a cell proliferation effect in the range of from 10 μM to 30 mM as compared to HSM alone.

HSM supplemented with sodium lactate was confirmed to have a cell proliferation effect in the range of from 3 μM to 30 mM as compared to HSM alone.

HSM supplemented with lactic acid was confirmed to have a cell proliferation effect in the range of from 3 μM to 100 mM as compared to HSM alone.

Further, it was confirmed that HSM supplemented with calcium lactate in the range of from 300 μM to 30 mM had a high cell proliferation effect.

### Example 3

(Optical Microscope Observation 1)

In the above-mentioned Examples, precipitates were formed in culture dishes in some cases. The formation of the precipitates inhibits contact between cells and medium to adversely affect long-term survival. Therefore, conditions under which the precipitates were not formed were investigated.

(Materials and Methods)

### 1. Preparation of Cells, Media, etc.

201B7 cells, ReproFF, HSM, and calcium lactate were prepared in the same manner as in Example 2.

### 2. Culture

5×10⁴ 201B7 cells were seeded to a 96-well plate coated with Matrigel. The next day, to HSM, calcium lactate was added at 3 μM, 10 μM, 30 μM, 100 μM, 300 μM, 1 mM, 3 mM, 10 mM, 30 mM, or 100 mM, followed by culture for 7 days.

The cells that had been cultured for 7 days were observed with an optical microscope (CKX41N-31PHP; Olympus, Tokyo, Japan).

(Results)

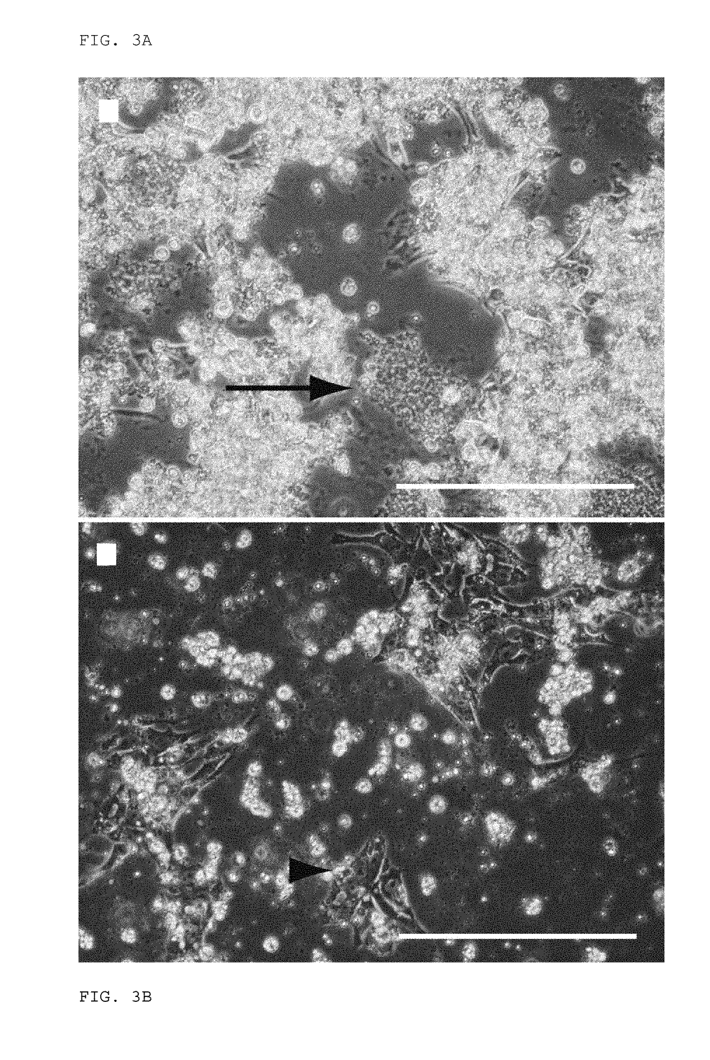

Typical results are shown in FIG. 3A and FIG. 3B.

As a result of the observation of the cells that had been cultured with calcium lactate for 7 days with an optical microscope, precipitates were found at 3 mM or more (the arrow of FIG. 3A). Meanwhile, precipitates were not found at 1 mM or less (the arrowhead of FIG. 3B).

In addition, at 1 mM of calcium lactate, the cells survived. HSM is supplemented with sodium bicarbonate so that sodium bicarbonate buffers the pH of the medium together with the 5% carbon dioxide gas of an incubator to keep the pH at about 7.4. At 3 mM or more of sodium lactate, a risk of precipitate formation through a reaction with sodium bicarbonate was conceivable.

Therefore, in the following Examples, the concentration of each of calcium lactate, sodium lactate, and lactic acid was set to 1 mM.

### Example 4

(Expressions of AFP and Albumin)

iPS cells were cultured in HSM supplemented with lactic acid, and the expressions of AFP and albumin in the culture cells were examined.

(Materials and Methods)

### 1. Preparation of Cells, Media, etc.

201B7 cells, ReproFF, HSM, calcium lactate, and sodium lactate were prepared in the same manner as in Example 2.

### 2. Culture

The 201B7 cells were seeded to a 6-well plate coated with Matrigel, and when 100% confluence had been reached, the medium was exchanged with 1 mM calcium lactate (Ca-lactate), 1 mM sodium lactate (Na-lactate), or 1 mM lactic acid (Lactate), followed by culture for 7 days. Control cells were cultured in ReproFF.

3. qPCR

RNA was extracted and analyzed for the expression levels of AFP and albumin by real-time quantitative PCR.

(Results)

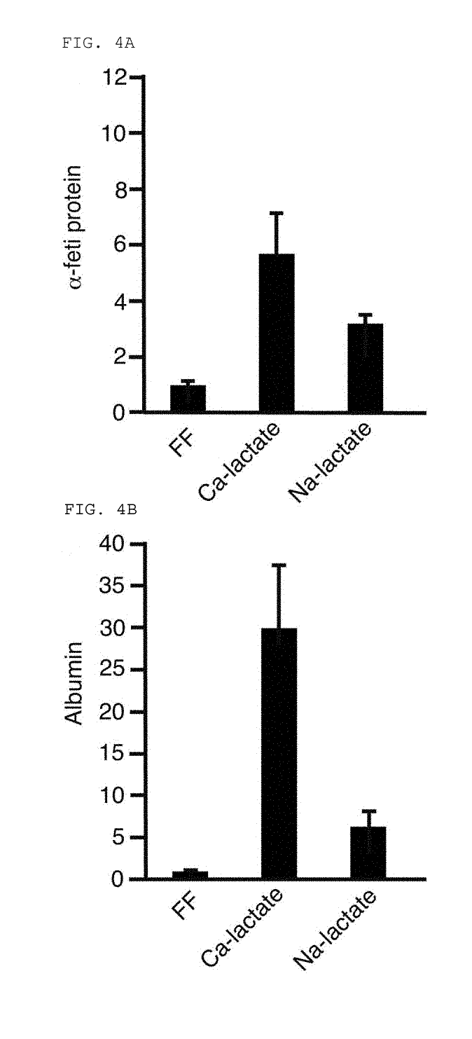

The results are shown in FIG. 4A and FIG. 4B. With calcium lactate and sodium lactate, the expressions of both AFP (FIG. 4A) and albumin (FIG. 4B) were upregulated as compared to those under the undifferentiated state.

Thus, through culture in HSM supplemented with calcium lactate or sodium lactate, the expressions of AFP and albumin serving as markers of hepatoblasts were upregulated, and hence it was confirmed that the induction of differentiation to hepatoblasts was promoted.

### Example 5

(Optical Microscope Observation 2)

iPS cells were cultured in HSM supplemented with lactic acid, and the state of the culture cells was confirmed.

(Materials and Methods)

### 1. Preparation of Cells, Media, etc.

201B7 cells, HSM, calcium lactate, sodium lactate, and lactic acid were prepared in the same manner as in Example 2.

### 2. Culture

The 201B7 cells were seeded to a 6-well plate coated with Matrigel, and when 100% confluence had been reached, the medium was exchanged with 1 mM calcium lactate, 1 mM sodium lactate, or 1 mM lactic acid, followed by culture for 7 days.

The cells that had been cultured for 7 days were observed with an optical microscope (CKX41N-31PHP; Olympus, Tokyo, Japan).

(Results)

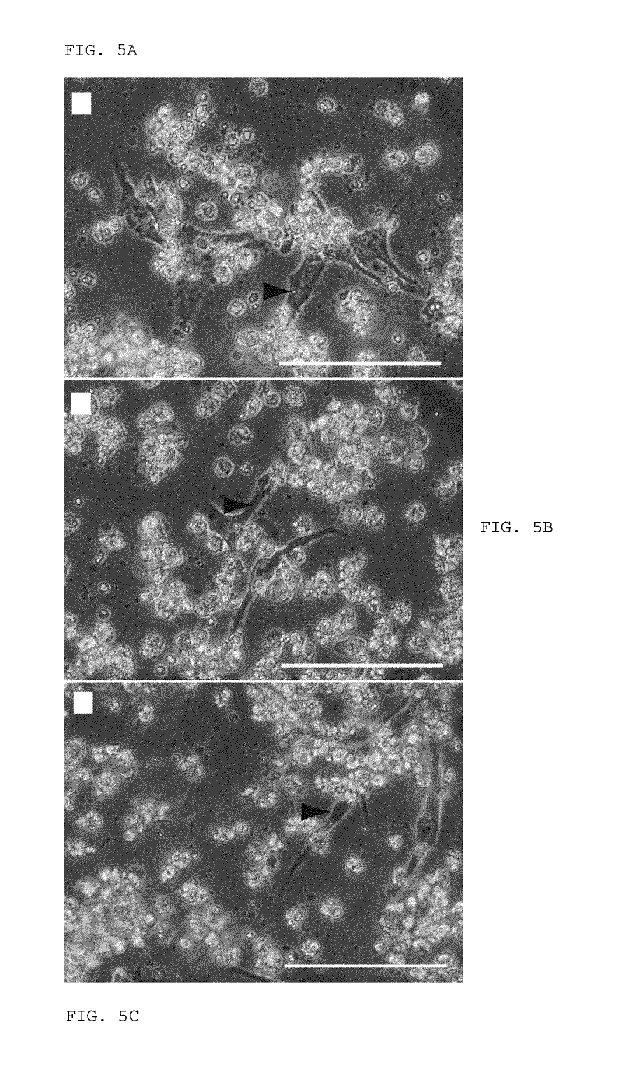

The results are shown in FIG. 5A, FIG. 5B, and FIG. 5C.

The cells cultured with sodium lactate (FIG. 5B) and lactic acid (FIG. 5C) were found to have a collapsing tendency, i.e., a tendency to be reduced in area of cytoplasm as compared to the cells cultured with calcium lactate (FIG. 5A) (the arrowheads in FIG. 5A, FIG. 5B, and FIG. 5C).

### Example 6

(Immunostaining)

iPS cells were cultured in HSM supplemented with lactic acid, and AFP expression in the culture cells was examined using an anti-AFP antibody.

(Materials and Methods)

### 1. Preparation of Cells, Media, etc.

201B7 cells, HSM, ReproFF, and calcium lactate were prepared in the same manner as in Example 2.

### 2. Culture

The 201B7 cells were seeded to an 8-well chamber slide and cultured. After 100% confluence had been reached, 1 mM calcium lactate was added to HSM, followed by culture for 7 days. As a control, the cells were cultured in ReproFF for 7 days.

### 3. Immunostaining

The cells were fixed with 4% paraformaldehyde, and washed with phosphate buffered saline (PBS). The cells were incubated at 4° C. for 30 minutes in 100% methanol having added thereto 0.3% hydrogen peroxide solution to inactivate intrinsic peroxidase. The cells were washed three times with PBS. The cells were incubated at 4° C. for 30 minutes in PBS supplemented with 2% fetal bovine serum (wash buffer).

A mouse anti-human alpha-fetoprotein (AFP) antibody (manufactured by Takara Bio Inc., product code: M225) was diluted 1,000-fold with the wash buffer and added, followed by incubation at 4° C. overnight. The resultant was washed three times with PBS. An anti-mouse IgG antibody (manufactured by GE Healthcare) labeled with Horse-radish peroxidase was diluted 1,000-fold with the wash buffer, followed by incubation at 4° C. for 3 hours. The resultant was washed three times with PBS. Color development was performed with 3,3-diaminobenzidine (DAKO, K3467), and nuclei were stained with hematoxylin, followed by mounting.

(Results)

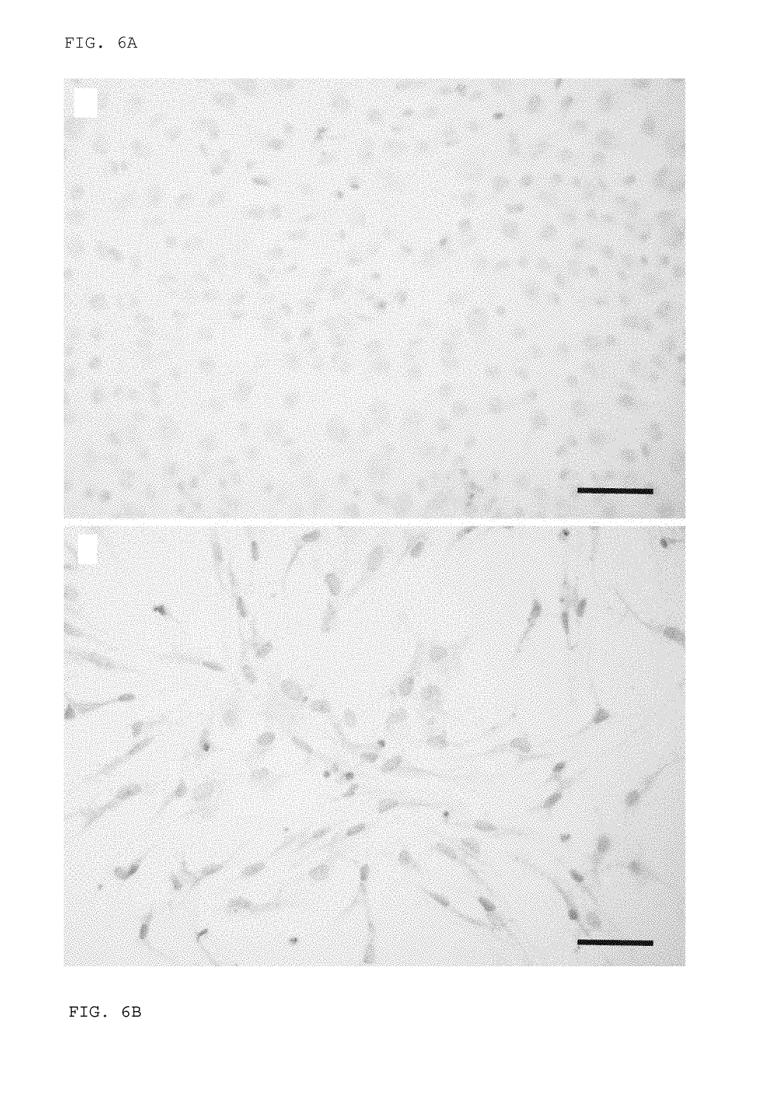

The results are shown in FIG. 6A and FIG. 6B. The result of the 7-day culture in HSM supplemented with lactic acid is shown in FIG. 6A, and the result of the 7-day culture in ReproFF is shown in FIG. 6B.

It was confirmed that the AFP expression was upregulated in the cells that had been cultured in HSM supplemented with lactic acid for 7 days as compared to the cells that had been cultured in ReproFF for 7 days.

Thus, through culture of iPS cells in HSM supplemented with lactic acid, the expressions of AFP and albumin serving as markers of hepatoblasts were upregulated, and hence it was confirmed that the induction of differentiation to hepatoblasts was promoted.

### Example 7

(Culture in HSM Supplemented with Pyruvic Acid)

It was confirmed from the results of Example 1 that, when pluripotent stem cells were cultured in HDI, the production of pyruvic acid was insufficient. HDI is obtained by adding agents and the like to HSM. In order to remove the influences of the agents added in HDI, iPS cells were cultured in HSM supplemented with pyruvic acid, and the efficiency of induction of hepatocytes from the iPS cells was investigated.

(Materials and Methods)

### 1. Preparation of Cells, Media, etc.

201B7 cells were used as the iPS cells in the same manner as in Example 1.

HSM medium was prepared in the same manner as in Example 1.

Sodium pyruvate (manufactured by Thermo Fisher Scientific) was used as a pyruvic acid source.

### 2. Culture

5×10⁴ 201B7 cells were seeded to a 96-well plate coated with Matrigel. The next day, to HSM, calcium lactate, sodium lactate, and lactic acid were each added at 3 μM, 10 μM, 30 μM, 100 μM, 300 μM, 1 mM, 3 mM, 10 mM, 30 mM, or 100 mM, followed by culture for 3 days. Control cells were cultured in ReproFF or HSM.

### 3. MTS Assay

After 3 days from the initiation of the culture, MTS assay (CellTiter 96 (registered trademark in Japan) AQueous One Solution, manufactured by Promega) was performed to investigate cell proliferation.

(Results)

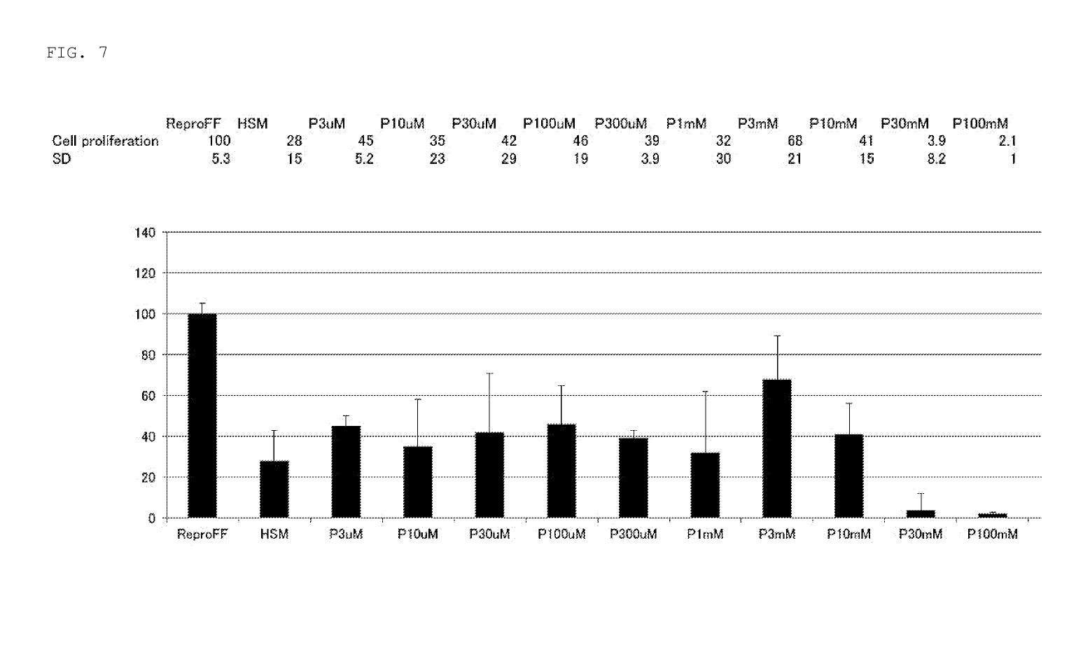

The results of the cell proliferation are shown in FIG. 7.

As apparent from FIG. 7, HSM supplemented with pyruvic acid was confirmed to have a cell proliferation effect in the range of from 3 μM to 10 mM as compared to HSM alone.

(General Remark)

The foregoing results revealed that, when iPS cells were cultured in HSM supplemented with lactic acid, unexpectedly, the cells survived even after 7 days. Further, surprisingly, it was revealed that, when iPS cells were cultured in HSM supplemented with lactic acid, the expressions of AFP and albumin were upregulated. That is, it is considered that lactic acid not only promotes the survival of iPS cells in HSM, but also promotes differentiation to hepatocytes.

The inventors of the present invention consider as described below.

Humans produce energy from glucose through glycolysis and the citric acid cycle. Vigorous exercise causes muscle to produce energy through glycolysis. When glucose goes through glycolysis, lactic acid is finally produced. Hepatocytes convert lactic acid to glucose through the Cori cycle. That is, lactic acid produced in muscle flows in blood to reach hepatocytes, and is converted to glucose by the hepatocytes before being released into the blood again. It is considered that, when lactic acid is added to HSM, the Cori cycle is activated to produce glucose. In the Cori cycle activation process, it is possible that differentiation to hepatocytes is promoted. Therefore, the inventors consider that hepatocytes can be produced within a short period of time by culturing pluripotent stem cells in HSM supplemented with lactic acid.

When hepatocytes can be induced to differentiate from human induced pluripotent stem (iPS) cells, application to a transplantation therapy for cases with liver failure, a drug toxicity test, and the like becomes possible.

1. A production method for hepatocyte lineage cells, comprising culturing pluripotent stem cells in a hepatocyte differentiation-inducing medium containing lactic acid or a salt thereof.

2. A production method according to claim 1, wherein the lactic acid or the salt thereof comprises one of calcium lactate, sodium lactate, and lactic acid.

3. A production method according to claim 1, wherein the pluripotent stem cells comprise induced pluripotent stem (iPS) cells.

4. A production method according to claim 1, wherein the hepatocyte lineage cells comprise hepatoblasts.

5. A production method for hepatoblasts, comprising culturing iPS in a hepatocyte differentiation-inducing medium containing calcium lactate, sodium lactate, or lactic acid.

6. A production method according to claim 1, wherein a concentration of the lactic acid or the salt thereof in the medium is from 1 μM to 100 mM.

7. A production method according to claim 1, wherein the lactic acid or the salt thereof in the medium comprises 5 μM to 65 mM calcium lactate.

8. A production method according to claim 1, wherein the lactic acid or the salt thereof in the medium comprises 300 μM to 30 mM calcium lactate.

9. A production method according to claim 1, wherein the lactic acid or the salt thereof in the medium comprises 5 μM to 2 mM calcium lactate.

10. A production method according to claim 1, wherein the lactic acid or the salt thereof in the medium comprises 300 μM to 2 mM calcium lactate.

11. A production method according to claim 1, wherein the lactic acid or the salt thereof in the medium comprises 1 μM to 50 mM sodium lactate.

12. A production method according to claim 1, wherein the lactic acid or the salt thereof in the medium comprises 1 μM to 100 mM lactic acid.

13. A production method according to claim 1, wherein the medium contains pyruvic acid or a salt thereof.

14. A production method according to claim 12, wherein a pyruvic acid concentration in the medium is from 1 μM to 20 mM.

15. A hepatocyte lineage cell, which is produced by the method of claim 1.

16. A cell culture product, which is produced by the method of claim 1.

17. A hepatocyte differentiation-inducing medium, comprising:
a composition shown in Table 1 below; and
lactic acid or a salt thereof at a lactic acid concentration of from 1 μM to 100 mM.
TABLE 1

| L15-ES medium | Inorganic salts | | |
|---|---|---|---|
| (1 L) | CaCl₂•2H₂O 1M solution | 1.258 | ml |
| | MgCl₂•6H₂O | 0.203 | g |
| | MgSO₄ (anhyd) | 0.098 | g |
| | KCl 1M solution | 5.3655 | ml |
| | KH₂PO₄ | 0.06 | g |
| | NaCl 5M solution | 27.29 | ml (7.915 g) |
| | NaH₂PO₄•2H₂O | 0.14014 | g |
| | Amino acids | | |
| | L-Alanine | 0.225 | g |
| | L-Arginine•HCl | 0 | g |
| | L-Asparagine•H₂O | 0.25 | g |
| | L-Cysteine | 0.12 | g |
| | L-Cystine•2HCl | 0 | g |
| | L-Glutamine | 0.3 | g |
| | Glycine | 0.2 | g |
| | L-Histidine•HCl•H₂O | 0.25 | g |
| | L-Isoleucine | 0.25 | g |
| | L-Leucine | 0.125 | g |
| | L-Lysine•HCl | 0.075 | g |
| | L-Methionine | 0.075 | g |
| | L-Phenylalanine | 0.125 | g |
| | L-Serine | 0.2 | g |
| | L-Threonine | 0.3 | g |
| | L-Tryptophan | 0.02 | g |
| | L-Tyrosine•2Na•H₂O | 0 | g |
| | L-Valine | 0.1 | g |
| | L-Ornithine•HCl | 0.169 | g |
| Gibco MEM | Vitamins | | |
| vitamins (diluted | Sodium chloride | 0.085 | g |
| 100-fold) 10 ml | Choline chloride | 0.001 | g |
| | Folic acid | 0.001 | g |
| | myo-Inositol | 0.001 | g |
| | Niacinamide | 0.001 | g |
| | D-Pantothenic acid•½Ca | 0.001 | g |
| | Pyridoxal•HCl | 0.001 | g |
| | Riboflavin | 0.0001 | g |
| | Thiamine•HCl | 0.001 | g |
| Others | Others | | |
| | D(+)-Galactose | 0.9 | g |
| | D-Glucose | 0 | g |
| | Phenol red•Na | 0.01 | g |
| | Glycerol (specific gravity: | 0.365 | ml |
| | 1.26 g/ml) | | |
| | Sodium pyruvate | 0 | ml |
| | Proline | 0.03 | g |
| | 7.5% Sodium hydrogen | 36.6 | ml |
| | carbonate solution | | |
| | Mercaptoethanol (10⁻⁴M | 1,000 | μl |
| | solution) | | |
| | Non-essential amino acids | 0 | ml |
